(19) United States
(12) Patent Application Publication
(10) Pub. No.: US 2026/0198460 A1
(43) Pub. Date: Jul. 16, 2026

(54) OLFACTOMETER

(71) Applicant: UNIVERSITY OF FLORIDA RESEARCH FOUNDATION, INCORPORATED, Gainesville, FL (US)

(72) Inventors: Thomson Paris, Gainesville, FL (US); Xavier Philippe Martini, Gainesville, FL (US); Robert Parsons, Tallahassee, FL (US)

(21) Appl. No.: 19/350,649

(22) Filed: Oct. 6, 2025

Related U.S. Application Data
(60) Provisional application No. 63/704,150, filed on Oct. 7, 2024.

(51) Int. Cl. A01K 29/00; G01N 33/00

(52) U.S. Cl. CPC A01K 29/005; G01N 33/0001

(57) ABSTRACT

An apparatus (100), including: a plurality of discrete scent generating assemblies (400), each configured to supply a respective flow of scented air; a mixer (500) having configured to receive the respective flows of air; to mix them together into a mixture; and to supply a plurality of flows of the mixture; optionally a plurality of visual cue light sources (700); and an arena assembly (600) having a plurality of discrete lanes (602), each lane configured to house an insect therein, to receive a respective flow of the mixture, and to receive visual cue light from a respective visual cue light source.

### STATEMENT REGARDING FEDERALLY SPONSORED DEVELOPMENT

This invention was made with government support under Grant No. 1821914 awarded by the National Science Foundation. The government has certain rights in the invention.

### SUPPORT STATEMENT

The invention was made whole or in part as a result of The Center for Arthropod Management Technologies a National Science Foundation Industry and University Cooperative Research Center.

### FIELD OF THE INVENTION

The invention relates to an olfactometer. In particular, the invention relates to an olfactometer suitable for use with insects, but which can be scaled to work with any animal, including humans.

### BACKGROUND

An olfactometer is an instrument used to detect and measure odor concentration, or to control the intensity of odors presented to test subjects. An olfactometer is a device that can precisely detect and measure odor concentrations at various points or can deliver odors of controlled intensity to test subjects. Alternatively, an olfactometer can be a device used for producing aromas in a precise and controlled manner. Olfactometers can also be used to gauge the odor detection threshold for various substances, or to determine odor preferences. Olfactometers are commonly used in conjunction with human subjects in laboratory settings, most often in market research, to quantify and qualify human olfaction. To measure intensity, conventional olfactometers generally introduce an odorous gas as a baseline against which other odors are compared, without a clear odor boundary demarcation.

Entomologists and other biologists also use olfactometry to understand the behavior of insects and similar organisms which use odors to search for nutrition, find mates, and avoid danger. Understanding which odors attract and repel insects allows scientists to control pests and to protect people, crops, and animals.

### BRIEF DESCRIPTION OF DRAWINGS

FIG. 1A to FIG. 1C are various views of an example embodiment of an insect olfactometer disclosed herein.

FIG. 2A and FIG. 2B show an example embodiment of a manifold of the insect olfactometer of FIG. 1.

FIG. 3 shows the insect olfactometer of FIG. 1 but with only one example embodiment of a scent generating assembly visible.

FIG. 4 is a sectional view of the insect olfactometer of FIG. 1.

FIG. 5A to FIG. 5F are various views of an example embodiment of an arena assembly of the insect olfactometer of FIG. 1.

FIG. 6A to FIG. 6B show an alternate example embodiment of an insect olfactometer disclosed herein.

FIG. 7A shows an example embodiment of a portion of an odor control lane of the insect olfactometer of FIG. 6A

FIG. 7B to FIG. 7E are various sectional views taken along various lines shown in FIG. 7A.

FIG. 8A to FIG. 8B show an example embodiment of a visual signal control lane of the insect olfactometer of FIG. 6A.

### DETAILED DESCRIPTION

The present inventors have devised a unique and innovative olfactometer that is configured to track animal responses to a variety of controllable olfactory cues and optionally simultaneously with a variety of visual cues. The olfactometer disclosed herein is for use with insects. However, it may be appropriately scaled to work with any animal, including humans.

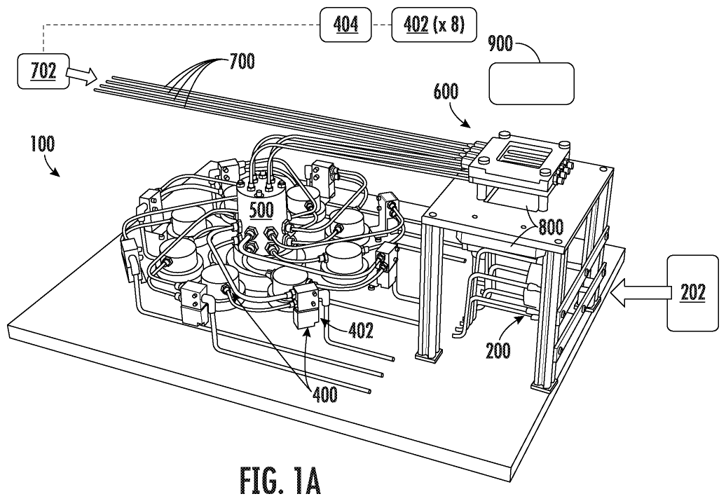

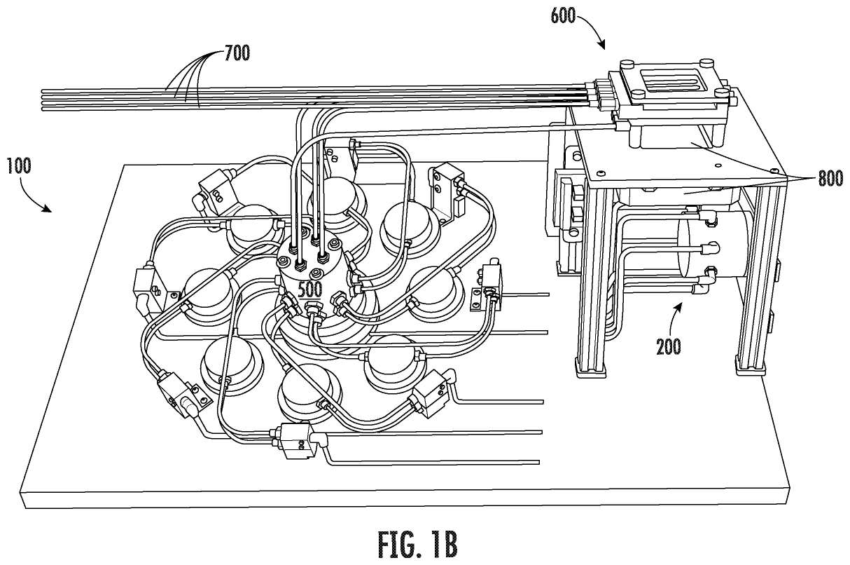

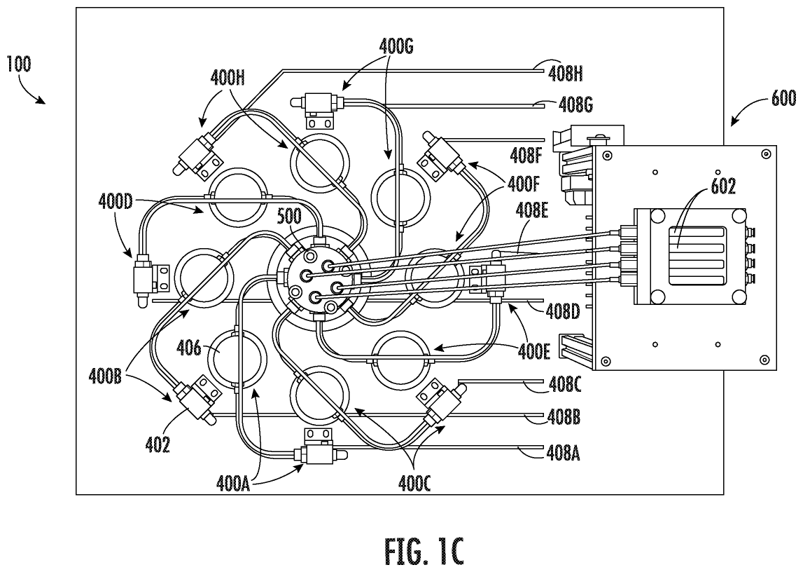

FIG. 1A to FIG. 1C are various views of an example embodiment of the insect olfactometer 100 disclosed herein.

In general terms, the (high throughput) insect olfactometer 100 includes a manifold 200 configured for fluid communication with a clean air supply 202. The manifold 200 is configured to create plural discrete flows of clean air. Each flow of clean air is delivered to a respective discrete scent generating assembly 400 of several scent generating assemblies 400 (e.g., eight shown). A respective flow controller 402 controls the flow of clean air through each scent generating assembly 400 and each flow controller 402 is individually controllable by a controller 404. Each scent generating assembly 400 is configured to generate a respective discrete flow of scented air and optionally a respective discrete flow of clean air.

A mixer 500 is configured to receive each flow of scented air and optionally each flow of clean air from each scent generating assembly 400. The mixer 500 mixes the flows it receives into a mixture and generates multiple discrete flows of the mixture to an arena assembly 600. The arena assembly 600 has multiple discrete lanes 602, each configured to house a respective insect and to receive a respective flow of the mixture. The insect olfactometer 100 further includes several visual cue light sources 700 (optionally emanating from a light assembly 702) and each lane 602 is further configured to receive visual cue light from a respective visual cue light source 700. Each lane is configured to cooperate with a silhouetting light source 800 to silhouette each insect and a tracking system 900 is used to monitor each silhouetted insect's response to various olfactory and visual cues presented to it.

Having a plurality of scent generating assemblies 400, each capable of generating its own unique scent and each individually controllable by a controller allows for great flexibility in olfactory testing. Having visual cue light delivered to each lane allows for testing of combined olfactory and visual cues, which greatly increases the testing capabilities of the insect olfactometer. Having individual lanes optionally olfactorily and visually isolated from the other lanes allows for greater testing control and thereby more accurate results.

As can be seen best in FIG. 1C, the insect olfactometer 100 includes plural scent generating assemblies 400A, 400B, 400C, 400D, 400E, 400F, 400G, 400H, each comprising a respective flow controller 402 and a respective vial assembly 406. For each scent generating assembly 400A, 400B, 400C, 400D, 400E, 400F, 400G, 400H, there is a respective clean air supply line 408A, 408B, 408C, 408D, 408E, 408F, 408G, 408H from the manifold 200.

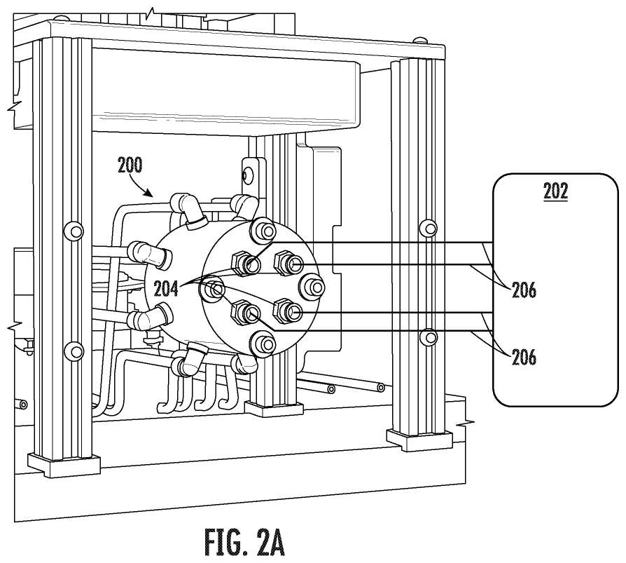

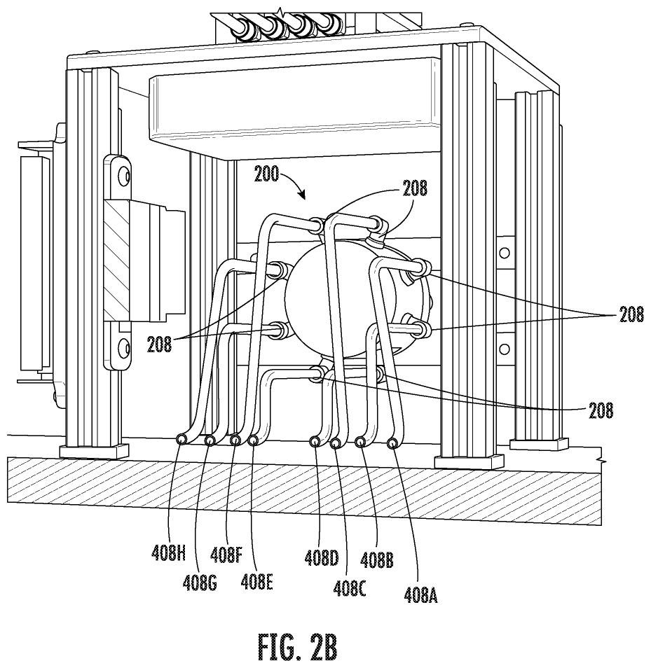

FIG. 2A shows an example embodiment of an inlet side of the manifold 200. The clean air supply 202 is in fluid communication with manifold inlets 204 and delivers clean air to the manifold inlets 204 via clean air supply lines 206.

FIG. 2B shows an example embodiment of an outlet side of the manifold 200. The manifold 200 is configured to take the clean air received from the clean air supply 202 and supply multiple discrete (eight shown) clean air flows via respective manifold outlets 208. Each manifold outlet 208 is associated with a respective clean air supply line 408A, 408B, 408C, 408D, 408E, 408F, 408G, 408H which leads to a respective scent generating assembly 400A, 400B, 400C, 400D, 400E, 400F, 400G, 400H. In an example embodiment, the clean air supply 202 delivers humidified air at 0.1 L/min. However, other clean air supply parameters are possible.

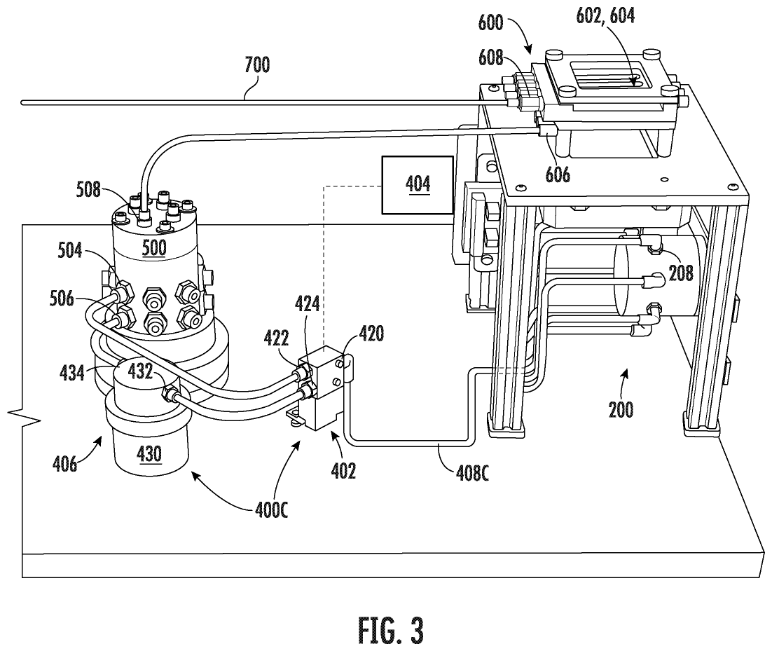

FIG. 3 shows the insect olfactometer 100 but with only one scent generating assembly 400C visible for simplicity. In an example embodiment, the structure and operation of the scent generating assembly 400C disclosed below is applicable to all the scent generating assemblies 400A, 400B, 400C, 400D, 400E, 400F, 400G, 400H. Minor changes may include the location of each as well as the specific routing of the respective clean air supply line 408A, 408B, 408C, 408D, 408E, 408F, 408G, 408H which leads to a respective scent generating assembly 400A, 400B, 400C, 400D, 400E, 400F, 400G, 400H. However, other differences may exist in alternate example embodiments.

The scent generating assembly 400C includes a (scent generating assembly) flow controller 402 and a vial assembly 406.

The flow controller 402 may be, for example, a solenoid or a controllable valve etc. that can be individually controlled by the controller 404 via wired or wireless signal communication. The flow controller 402 has a flow controller clean air inlet 420 in fluid communication with the clean air supply line 408C, a flow controller first clean air outlet 422 that leads to the mixer 500, and a flow controller second clean air outlet 424 that leads to the vial assembly 406. When the flow controller 402 is open, a flow of clean air from the flow controller clean air inlet 420 to the flow controller first clean air outlet 422 and a flow of clean air from the flow controller clean air inlet 420 to the flow controller second clean air outlet 424 are enabled. When the flow controller is closed the flow of clean air from the flow controller clean air inlet 420 to the flow controller first clean air outlet 422 and the flow of clean air from the flow controller clean air inlet 420 to the flow controller second clean air outlet 424 are stopped.

Since the flow controller first clean air outlet 422 leads to the mixer 500 and the flow controller second clean air outlet 424 leads to the vial assembly 406, when the flow controller 402 is open the flow of clean air to the vial assembly 406 and the flow of clean air to the mixer 500 are enabled. When the flow controller 402 is closed the flow of clean air to the vial assembly 406 and the flow of clean air to the mixer 500 are stopped.

The vial assembly 406 includes a vial chamber 430, a vial chamber clean air inlet 432, and a vial chamber outlet 434. The vial chamber clean air inlet 432 is in fluid communication with the flow controller second clean air outlet 424, receives a flow of clean air therefrom when the flow controller 402 is open, and opens into the vial chamber 430. The vial chamber 430 is configured to contain a solid or a fluid having one or more odorants or a solvent control etc. Each of the vial assemblies 406 can have a distinct scent/odorant and/or a mixture thereof. The clean air entering the vial chamber 430 interacts with the solid or the fluid to create a flow of scented air that exits the chamber via the vial chamber outlet 434. The vial chamber outlet 434 leads to the mixer 500.

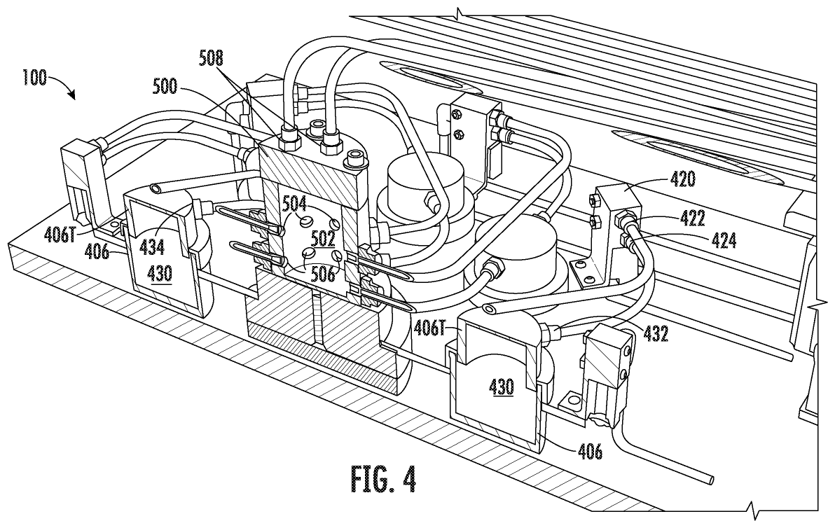

The mixer 500 includes a mixing chamber 502 (FIG. 4), a plurality of mixing chamber first inlets 504, a plurality of mixing chamber second inlets 506, and a plurality of mixing chamber outlets 508. Each mixing chamber first inlet 504 is in fluid communication with a respective flow controller first clean air outlet 422, receives a flow of clean air therefrom when the flow controller 402 is open, and opens into the mixing chamber 502. Each mixing chamber second inlet 506 is in fluid communication with a respective vial chamber outlet 434, receives a flow of scented air therefrom when the flow controller 402 is open, and opens into the mixing chamber 502.

Inside the mixing chamber 502, the flow(s) of clean air from the plurality of mixing chamber first inlets 504 and the flow(s) of scented air from the plurality of mixing chamber second inlets 506 are mixed to form a mixture. Since the flow controllers 402 are individually controllable by the controller 404, anywhere from none to all of the flow controllers 402 may be open. Only the respective scented flows from scent generating assemblies 400A, 400B, 400C, 400D, 400E, 400F, 400G, 400H with open flow controllers 402 are mixed in the mixer 500. Scented flows from the scent generating assemblies 400A, 400B, 400C, 400D, 400E, 400F, 400G, 400H will be sent individually or in combination to the arena assembly 600 according to the programming sequence determined by an operator and implemented via the controller 404.

The mixture formed in the mixing chamber 502 exits the mixing chamber via a plurality of mixing chamber outlets 508. Each mixing chamber outlet 508 leads to a respective lane 602 in the arena assembly 600. Each lane 602 thereby receives its own discrete flow of the (same) mixture.

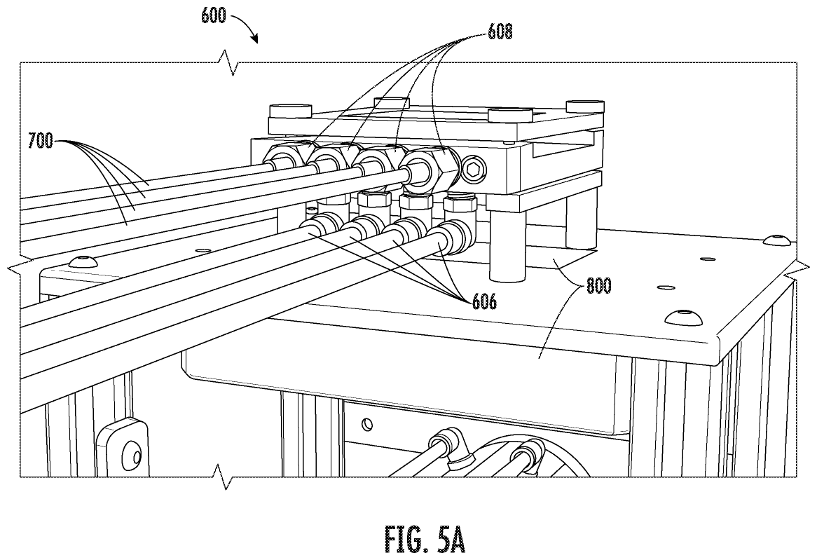

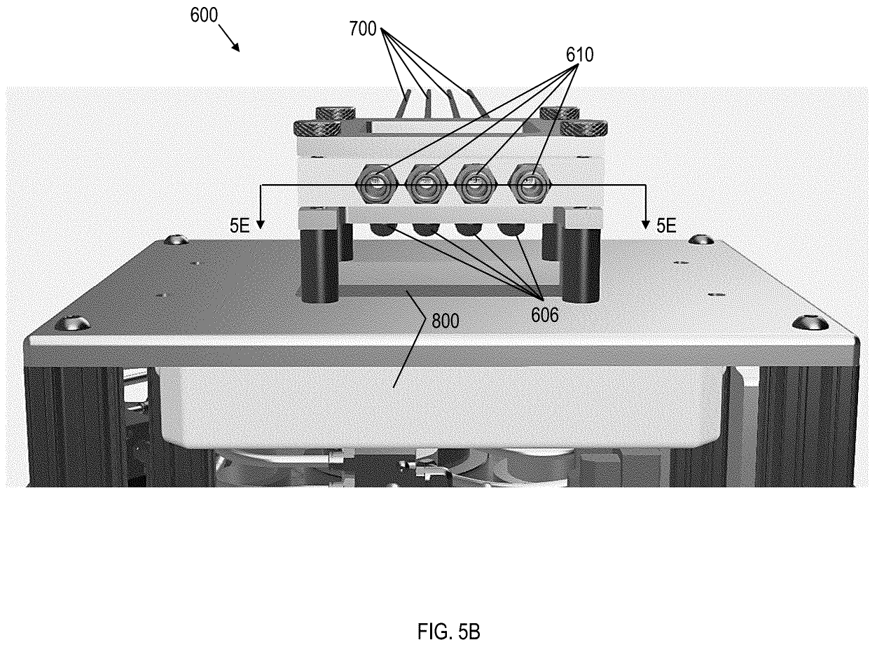

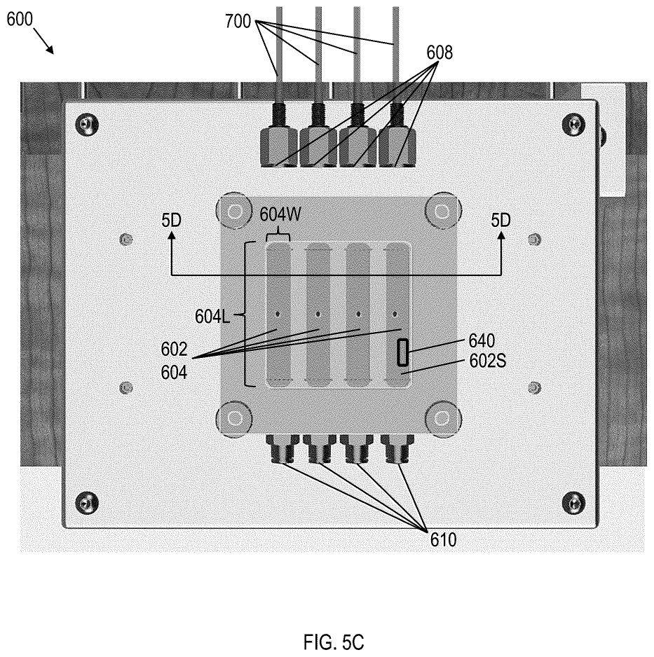

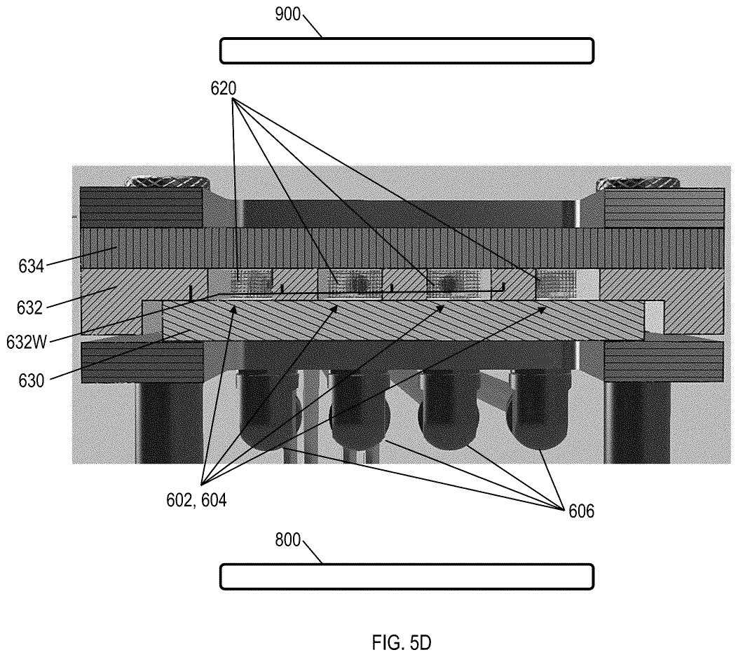

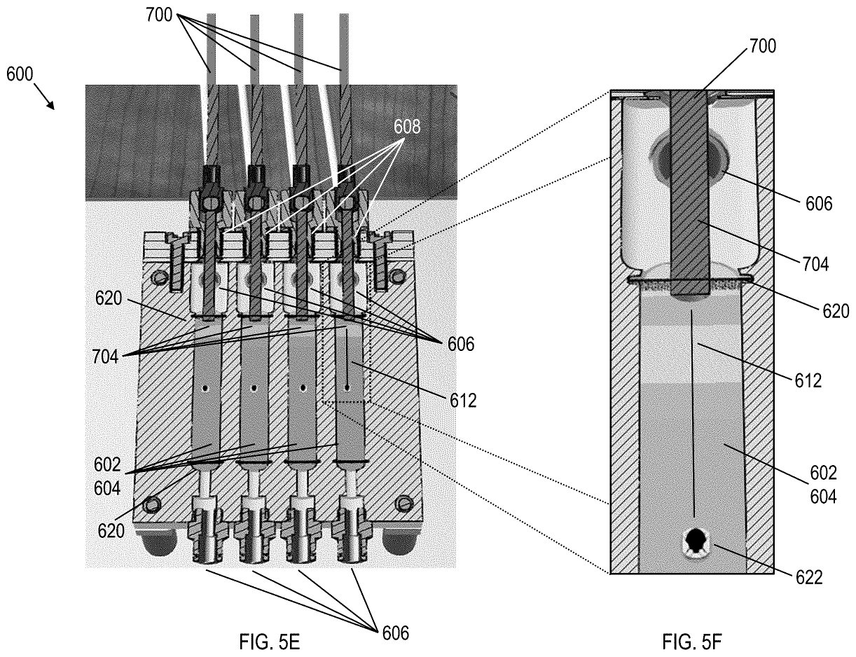

The lane 602 has an insect chamber 604 configured to house an insect therein (FIG. 5A to FIG. 5F) and an insect chamber inlet 606 in fluid communication with the mixing chamber outlet 508 which supplies the insect chamber 604 with a flow of the mixture.

FIG. 4 is a sectional view of two vial assemblies 406 and the mixer 500 of the insect olfactometer 100 of FIG. 1.

On the right, a flow controller second clean air outlet 424 leading to a vial chamber clean air inlet 432 and a vial chamber 430 of the vial assembly 406 are visible. On the left, a vial chamber 430 and a vial chamber outlet 434 are visible. The vial chamber outlet 434 leads to the mixing chamber second inlet 506 and to the mixing chamber 502. The mixing chamber outlets 508 lead to the arena assembly 600. The vial assemblies 406 may be sealed with a standard or custom-fitted aluminum top 406T, check valves (not shown) at the inlets and outlets, and a rubber O-ring (not shown). In an example embodiment, the mixer 500 can deliver pulses of the mixture (of the flows of scented air from up to eight (8) of the scent generating assemblies 400A, 400B, 400C, 400D, 400E, 400F, 400G, 400H) every ninety (90) seconds for 500 milliseconds after a thirty (30) minute adaptation period for the insect.

FIG. 5A to FIG. 5F are various views of an example embodiment of the arena assembly 600 of the insect olfactometer 100 of FIG. 1. FIG. 5D is a sectional view along line 5D-5D of FIG. 5C. FIG. 5E is a sectional view along line 5E-5E of FIG. 5B. FIG. 5F is a close-up view of a portion of a lane 602 of FIG. 5E.

The arena assembly 600 includes a plurality of lanes 602, each having an insect chamber 604, an insect chamber inlet 606 in fluid communication with a respective mixing chamber outlet 508 to supply a respective flow of the mixture, and an insect chamber outlet 610 to exhaust the respective flow of the mixture. Each lane 602 also has a visual cue light input 608 in light communication with a respective visual cue light source 700 as well as an optional collimator 704 to collimate the visual cue light and align the collimated light with a longitudinal axis 612 of the lane 602. The collimator 704 may be part of the visual cue light input 608 or may be separate. Optional screens 620 may be placed at ends of each lane 602 to keep the insect 622 inside the insect chamber 604.

The visual cue light sources 700 may be 1-to-4 fan-out fiber optic bundles with Ø600 μm core sizes connected to the light assembly 702 at one end and connected to a respective lane 602 at the other end via subminiature version A (SMA) connectors. The light assembly 702 may include a T-mount motorized shutter located in front of a light source within the light assembly 702. The motorized shutter may be digitally controlled by the controller 404 in wired or wireless signal communication therewith. The fiber optic bundles can transmit wavelengths ranging from deep UV 220 nm to over 750 nm.

The arena assembly 600 may be composed of several components, including (in a non-limiting example), a bottom piece 630, a middle piece 632, and a top piece 634. The middle piece may define walls 632W of the insect chambers 604. The bottom piece 630, the middle piece 632, and the top piece 634 may optionally be assembled together in such a way as to isolate the olfactory input and the light input to one insect chamber 604 from bleeding over to other insect chambers 604 (e.g., airtight and light tight). In an example embodiment, a lane length 604L is 6.3 centimeters (+/−0.1 centimeters) and a lane width 604W is 1.0 centimeters (+/−0.1 centimeters). However, the lane length 604L and the lane width 604W can be scaled to any size as desired.

One or more of the lanes 602 may be a sensor lane 602S having a sensor assembly 640 having one or more of a photoionization device, one or more photocells, a flow meter, a temperature sensor, a relative humidity sensor, and a fitting configured to cooperate with a solid phase microextraction (SPME) process. The photoionization device can measure the volatile organic compounds (in the mixture) that are pulsed through the mixer 500 into the insect chamber 604 where insects 622 are active and responding to odors. The one or more photocells can detect wavelengths in the ultraviolet and visible spectrum (e.g., 220 nm-750 nm) directed into the insect chamber 604. These and the other sensors will verify that the odors and visual cues are being sent into the insect chambers 604 according to the directives of the software developed to run the insect olfactometer 100 via the controller 404. In an example embodiment, a second SMA connector is installed on another side of the light assembly 702, with a single fiber optic cable linking to the sensor lane 602S.

The bottom piece 630 may be a floor for the insect chambers 604 and may be transparent or translucent (e.g., frosted, to scatter the light). The top piece 634 may be a ceiling for the insect chambers 604 and may also be transparent or translucent. The transparent or translucent property of the bottom piece 630 and the top piece 634 allow silhouetting light from the silhouetting light source 800 (e.g., infrared light) to shine upward through the bottom piece, 630, through the insect chambers 604, through the top piece 634, and to the tracking system 900 above the insect chambers 604. Any autonomous tracking system known to the artisan can be utilized (e.g., Noldus's Ethovision).

The tracking system 900 is used to individually monitor each insect's 622 response to various olfactory and visual cues presented to it (e.g., time resolution of approximately thirty (30) hertz). For each run, movements before, during, and after exposure can be quantified in one hundred (100) millisecond intervals, and response patterns can be visually represented in ethograms for individual insects repeatedly tested with various compounds. As a result, very precise and subtle responses to odor can be tracked and recorded.

Moreover, the olfactometer 100 disclosed herein may be appropriately scaled and used with other animals such as mice etc.

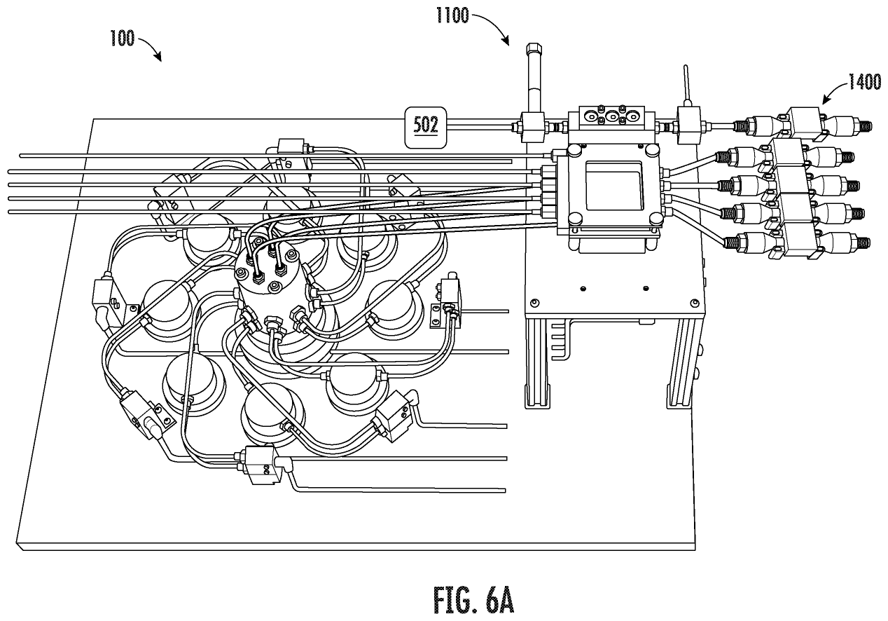

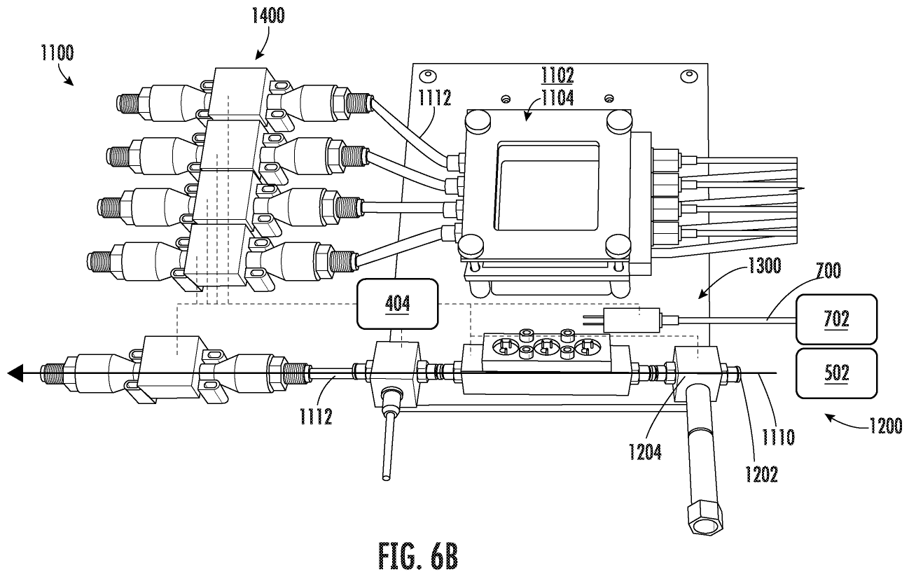

FIG. 6A to FIG. 6B show an alternate example embodiment of an insect olfactometer 1000. This example embodiment of the insect olfactometer 1000 functions like the insect olfactometer 100 of FIG. 1A except that this insect olfactometer 1000 includes a different arena assembly 1100. The arena assembly 1100 includes the conventional lanes 1102 as well as a separate odor control lane 1200 having respective odor control sensors and a separate visual signal control lane 1300 having respective visual signal sensors. An airflow sensor 1400 of each lane 1102, 1200 is also shown. The odor control lane 1200 is configured to precisely calibrate, control, and measure odor concentrations. Every sensor/component of the arena assembly 1100 may be monitored and/or controlled by the controller 404 and software thereon.

A conduit 1110 provides fluid communication between the mixing chamber 502 and an odor control lane inlet 1202 so the mixture from the mixing chamber 502 can then flow along a flow path 1204 through the odor control lane 1200. Conduits 1112 provide fluid communication between insect clambers 1106 of each lane 1102 and a respective airflow sensor 1400 of the respective lane 1102. A conduit 1112 likewise provides fluid communication to the airflow sensor 1400 of the odor control lane 1200. An example airflow sensor 144 is a Honeywell AWM5104VN sensor.

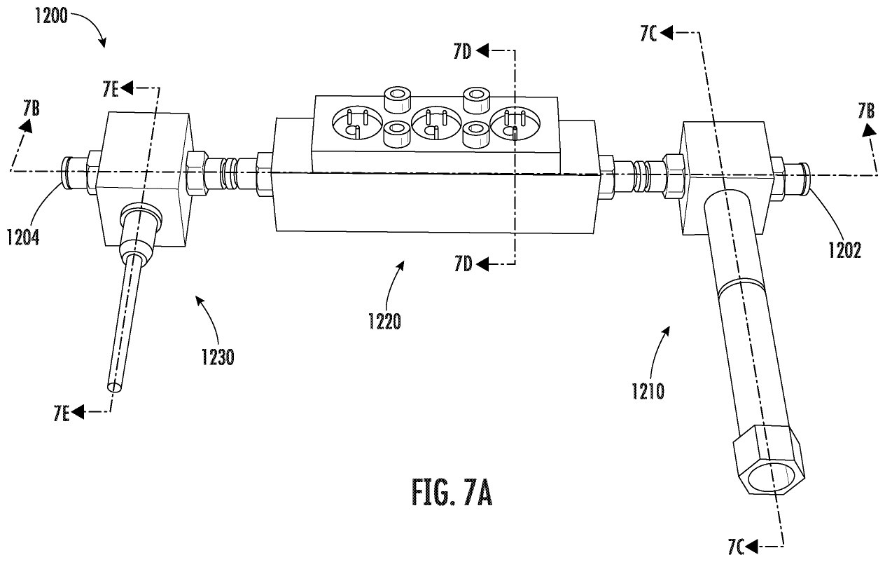

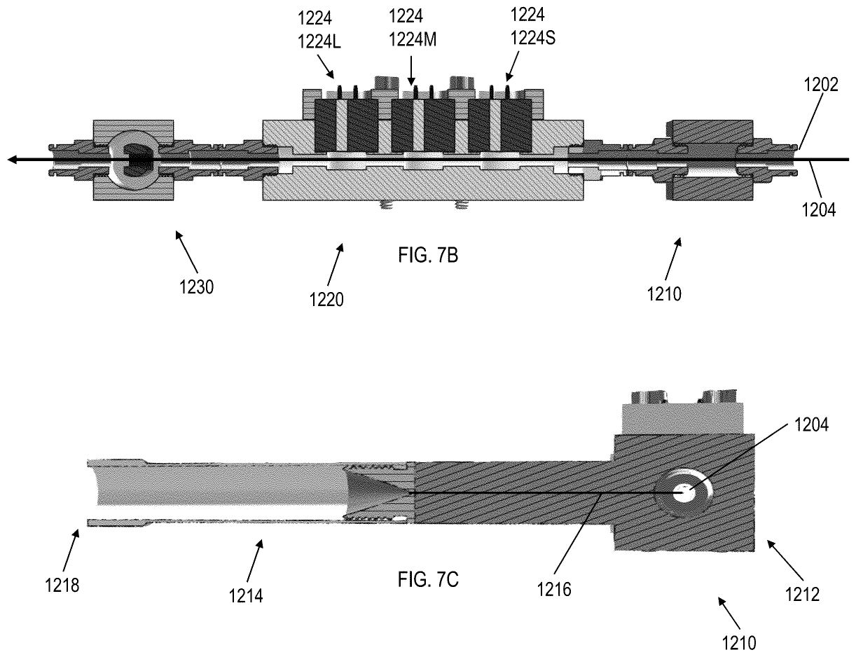

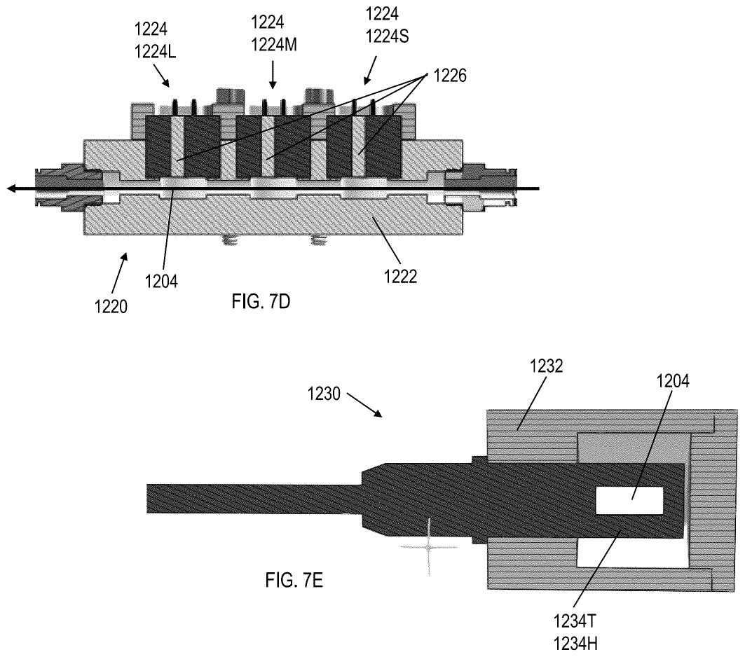

FIG. 7A shows a portion of the odor control lane 1200 without the respective airflow sensor 1400. The odor control lane 1200 can include a variety of sensors. In this example embodiment, the sensors include a solid phase microextraction fiber assembly 1210 (SPME assembly 1210), a VOC sensor positioning assembly 1220, and a relative temperature sensor and humidity sensor assembly 1230.

FIG. 7B is a sectional view along line 7B-7B in FIG. 7A showing the flow path 1204 through the portion of the odor control lane 1200.

FIG. 7C is a sectional view of the SPME assembly 1210 along line 7C-7C in FIG. 7A. The SPME assembly 1210 includes a body 1212, a holder assembly 1214, a needle 1216, and a port 1218. The flow path 1204 flows through the body 1212. The holder assembly 1214 is secured to the body 1212, positions the needle 1216 in the flow path 1204, and provides the access port 1218. The access port 1218 provides for connecting of SMPE fibers used for sampling.

FIG. 7D is a sectional view of the VOC sensor positioning assembly 1220 along line 7D-7D in FIG. 7A. The VOC sensor positioning assembly 1220 has a body 1222 through which the flow path 1204 flows and the body 1222 includes multiple ports 1224 at various positions along the flow path 1204. A VOC sensor 1226 can be positioned in any of the ports 1224 such that the VOC sensor 1226 will be in fluid communication with the flow path 1204. Typically, there may be only one VOC sensor 1226 present in one of the ports 1224. Ports 1224 without a VOC sensor 1226 may be plugged with, for example, Teflon® plugs and gaskets. An example VOC sensor 1226 is an Amphenol SGX Sensortech PID-10.6eV-10KB sensor.

FIG. 7E is a sectional view of the relative temperature sensor and humidity sensor assembly 1230 along line 7E-7E in FIG. 7A. The relative temperature sensor and humidity sensor assembly 1230 includes a body 1232 and a relative temperature sensor 1234T and a humidity sensor 1234H secured to the body 1232 and in fluid communication with the flow path 1204 through the body 1232.

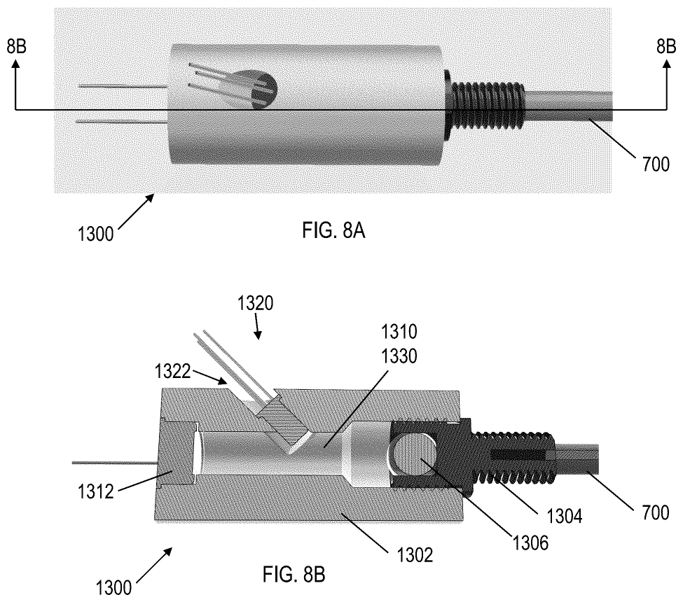

FIG. 8A shows an example embodiment of a visual signal control lane 1300. FIG. 8B is a sectional view of the visual signal control lane 1300 along line 8B-8B in FIG. 8A. The visual signal control lane 1300 is connected to a visual cue light source 700. The visual cue light source 700 delivers visual cue light to the visual signal control lane 1300 from the light assembly 702. In this example embodiment, the visual cue light source 700 may be 1-to-5 fan-out fiber optic bundles connected to the light assembly 702 at one end and connected to a respective lane 602 and to the visual signal control lane 1300 at the other end.

The visual signal control lane 1300 includes a body 1302 connected to the visual cue light source 700 via an adapter 1304 that houses a collimator 1306. The body 1302 defines a light-sealed chamber 1310 into which the collimator 1306 emits the visual cue light. The body 1302 further houses one or more light sensors 1312. The one or more light sensors 1312 may be, for example, an ultraviolet light sensor and/or an ultraviolet-visible light sensor. An example ultraviolet light sensor is an Advanced Photonix 008-2151-112, 220 nm-370 nm photodiode sensor. An example ultraviolet-visible light sensor is a PDV-C 406, 320 nm-850 nm photodiode sensor. In this example embodiment, two light sensors are used. Light sensor 1312 is the ultraviolet-visible light sensor, and a second light sensor 1320 is disposed in a port 1322. The second light sensor is the ultraviolet light sensor. However, the sensor positions may be swapped, different light sensors that cover different ranges may be used, and more or fewer light sensors may be used. A surface 1330 of the main body 1302 that defines the chamber 1310 may optionally be polished aluminum to enhance reflection of the visual cue light. This, in turn, may help the one or more light sensors 1312, 1320 to capture the visual cue light.

To evaluate the response of studied animals to odor pulses and provide detailed information on stimulus characteristics, the VOC sensor 1226 (e.g., a photoionization device (PID)) analyzes odor pulses of the mixture from the mixing chamber 502 within the odor control lane 1200. The VOC sensor 1226 detects and quantifies non-air volatiles with high temporal resolution. Additionally, the light sensors 1312 analyze the photo pulses (pulses of visual cue light) with high sensitivity. The controller 404 and software thereon process the analog input from both the VOC sensor 1226 and the light sensors 1312, representing the processed signals over the elapsed time.

To calibrate the speed of movement of the mixture from the mixing chamber 502, the distance from the odor control lane inlet 1202 to the VOC sensor 1226 can be adjusted by changing which port 1224 is used. The VOC sensor 1226 (or multiple VOC sensors 1226) can be positioned at various ports 1224S, 1224M, 1224L within the odor control lane 1200 to measure short, middle, and longest distances respectively from the odor control lane inlet 1202, which corresponds to where the insects/animals respond to the odors. The airflow sensors 1400 can be used in the odor control lane 1200 to ensure that the airspeed matches that of the lanes 602 containing the insects/animals.

As has been disclosed above, the present inventors have devised an apparatus with features that are improvements in the art. All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent, or similar purpose, unless expressly stated otherwise.

While various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims.

What is claimed is:

1. An apparatus, comprising:
a plurality of discrete scent generating assemblies, each comprising a vial assembly comprising: vial chamber; a vial chamber clean air inlet; and a vial chamber outlet;
a mixer comprising: a mixing chamber; a plurality of mixing chamber inlets, each mixing chamber inlet in fluid communication with a respective vial chamber output; and a plurality of mixing chamber outlets; and
an arena assembly, comprising: a plurality of discrete lanes, each comprising an insect chamber configured to house an insect therein; and an insect chamber inlet in fluid communication with a respective mixing chamber outlet.

2. The apparatus of claim 1,
further comprising a plurality of visual cue light sources;
wherein each lane further comprises a visual cue light input in light communication with a respective visual cue light source and with the insect chamber.

3. The apparatus of claim 2, wherein the visual cue light sources are configured to emit light in wavelengths from 220 nm to at least 750 nm.

4. The apparatus of claim 2, wherein each lane further comprises a collimator configured to align visual cue light from the respective visual cue light source with a longitudinal axis of the insect chamber.

5. The apparatus of claim 1,
wherein each lane comprises a translucent or transparent floor; and
wherein the arena assembly further comprises a silhouetting light source configured to emit silhouetting light into each lane through the translucent or transparent floor.

6. The apparatus of claim 5, wherein the silhouetting light comprises an infrared light.

7. The apparatus of claim 5, further comprising a tracking system configured to cooperate with the silhouetting light to track positions of respective insects in the plurality of discrete lanes.

8. The apparatus of claim 1, wherein each scent generating assembly further comprises a respective flow controller configured to selectively enable and disable a flow of clean air to the vial chamber clean air inlet.

9. The apparatus of claim 8, further comprising a controller configured to individually control each of the respective flow controllers.

10. The apparatus of claim 1,
wherein each scent generating assembly further comprises a scent generating assembly clean air outlet; and
wherein the mixer further comprises a plurality of mixing chamber clean air inlets, each in fluid communication with a respective scent generating assembly clean air outlet.

11. The apparatus of claim 10, wherein each scent generating assembly further comprises a respective scent generating assembly flow controller, wherein when the scent generating assembly flow controller is open a flow of clean air to the vial chamber clean air inlet and to the scent generating assembly clean air outlet is enabled, and when the scent generating assembly flow controller is closed the flow of clean air to the vial chamber clean air inlet and to the scent generating assembly clean air outlet is stopped.

12. The apparatus of claim 1, further comprising:
a manifold comprising: a clean air inlet configured for fluid communication with a clean air supply; and a plurality of manifold clean air outlets, each in fluid communication with the vial chamber clean air inlet of a respective discrete scent generating assembly.

13. The apparatus of claim 11, further comprising:
a manifold comprising: a clean air inlet configured for fluid communication with a clean air supply; and a plurality of manifold clean air outlets, each in fluid communication with a respective scent generating assembly flow controller.

14. The apparatus of claim 1, further comprising an odor control lane in fluid communication with the mixing chamber and comprising at least one of a solid phase microextraction assembly, a VOC sensor, an airflow sensor, a temperature sensor, and a relative humidity sensor.

15. The apparatus of claim 2, further comprising a visual signal control lane in light communication with a visual cue light source of the plurality of visual cue light sources and comprising at least one of a collimator, a light-sealed chamber, and a light sensor.

16. An apparatus, comprising:
a plurality of discrete scent generating assemblies, each configured to supply a respective flow of scented air;
a mixer configured to receive the respective flows of air; to mix them together into a mixture; and to supply a plurality of flows of the mixture;
a plurality of visual cue light sources; and
an arena assembly comprising a plurality of discrete lanes, each lane configured to house an insect therein, to receive a respective flow of the mixture, and to receive visual cue light from a respective visual cue light source.

17. The apparatus of claim 16, each lane further comprising a respective collimator configured to collimate the visual cue light entering the lane.

18. The apparatus of claim 16,
wherein each scent generating assembly is further configured to supply a respective flow of clean air; and
wherein the mixer is configured to receive each respective flow of clean air and to mix the respective flows of clean air into the mixture.

19. The apparatus of claim 16,
wherein each scent generating assembly is further configured to receive a respective flow of clean air; and
wherein each scent generating assembly further comprises a respective flow controller configured to selectively enable and disable the respective flow of clean air.

20. The apparatus of claim 19, further comprising a controller configured to individually control each of the respective flow controllers.
